(19) United States
(12) Patent Application Publication
(10) Pub. No.: US 2016/0024149 A1
(43) Pub. Date: Jan. 28, 2016

(54) BIOACTIVE MOLECULES PRODUCED BY PROBIOTIC BACTERIA AND METHODS FOR ISOLATING AND USING THE SAME

(71) Applicant: Trustees of Dartmouth College, Hanover, NH (US)

(72) Inventor: Ruth I. Connor, Hartland, VT (US)

(21) Appl. No.: 14/876,277

(22) Filed: Oct. 6, 2015

Related U.S. Application Data
(63) Continuation-in-part of application No. 12/937,417, filed on Oct. 12, 2010, now abandoned.
(60) Provisional application No. 61/045,779, filed on Apr. 17, 2008.

(51) Int. Cl. C07K 1/36; C12P 21/00; A61K 31/375; C07K 4/04; A61K 38/03

(52) U.S. Cl. CPC C07K 1/36; C07K 4/04; A61K 38/03; A61K 31/375; C12P 21/00

(57) ABSTRACT

The present invention is a method for isolating bioactive molecules secreted by probiotic bacteria such as Lactobacillus rhamnosus, and methods for using such bioactive molecules to activate NOD2, decrease expression of inflammatory molecules, inhibit replication of human immunodeficiency virus (HIV), stimulate expression of Apolipoprotein A-IV, modulate diet-associated weight gain and prevent mucosal transmission of HIV.

This application is a continuation-in-part of U.S. patent application Ser. No. 12/937,417, filed Oct. 12, 2010, which is the U.S. National Stage of PCT/US2009/036451, filed Mar. 9, 2009 which claims benefit of priority from U.S. Provisional Patent Application Ser. No. 61/045,779, filed Apr. 17, 2008, the contents of each of which are incorporated herein by reference in their entireties.

This invention was made with government support under Grant Nos. R21AI065235-02 and 1R21AI071948-01A1 awarded by the National Institutes of Health. The government has certain rights in the invention.

### INTRODUCTION

### Background of the Invention

Probiotics are live microorganisms which, when administered in adequate amounts, confer a health benefit to the host. Probiotics most commonly include strains of lactic acid bacteria within the genera of Lactobacillus and Bifidobacteria. In clinical trials, ingestion of live probiotic bacteria is associated with improvement in intestinal and immune health when probiotic products are consumed on a regular basis.

Health benefits conferred by probiotics are presumed to be dependent on maintenance and delivery of viable bacteria in adequate doses. However, live probiotics are subject to loss of viability and compromised quality due to exposure of the bacteria to moisture, heat, and changes in pH during manufacturing and storage. These conditions limit the inclusion of probiotic organisms in consumer products and therapeutics.

Culture filtrate from probiotic bacteria has been suggested for use in the treatment of disease. In this regard, Japanese Patent number JP63316726 suggests a polysaccharide or a protein polysaccharide produced by a bacterium belonging to Procaryomycota as an antiviral agent for suppressing infection of a retrovirus.

U.S. patent application Ser. No. 10/878,411 teaches a method of treating a mammal having a retroviral infection by administering a therapeutic agent composed of a concentrate of a filtrate of a culture solution obtained by co-culturing a particular combination of different types of beneficial organisms selected from yeasts and lactic acid bacteria.

U.S. patent application Ser. No. 10/413,993 teaches a method of preventing and treating viral infections or inhibiting the spread of viruses by administering compositions of at least one Lactobacillus whole cell or by-product thereof to patients in need of such treatment.

U.S. patent application Ser. No. 10/831,070 teaches L. acidophilus cell wall, cell surface and secreted proteins and fragments and variants thereof for use in preventing or reducing the occurrence of an infection in a host.

Silva, et al. (1987) Antimicrob. Agents Chemother. 31(8):1231-3) teach the presence of a low molecular weight compound (<1000 Da) secreted by LGG, that is heat stable, active between pH 3-5, distinct from lactic and acetic acids, and soluble in acetone water. It is suggested that this compound may be a short-chain fatty acid or microcin.

Tao, et al. (2006) Am. J. Physiol. Cell Physiol. 290(4):C1018-30) teach that soluble factors produced by LGG activate MAPK and induce heat shock proteins in intestinal epithelial cells. These effects are mediated by a low-molecular weight peptide that is heat and acid stable, protease sensitive, and <10 kDa.

Sjogren ((2005) Doctoral Thesis, Swedish University of Agricultural Sciences) describes the use of porous graphitized carbon to separate antifungal metabolites present in cell-free supernatants of strains of lactic acid bacteria and propionic acid bacteria such as Lactobacillus sp., Pediococcus sp., and Propionibacterium sp.

Bajad, et al. ((2006) J. Chromatograph. A 1125:76-88) describe metabolome analysis of E. coli. This reference teaches culturing E. coli in minimal salts media and carbon-starvation conditions, extracting metabolites from the E. coli and the use of porous graphitized carbon to separate and evaluate metabolites.

WO 84/00777 teaches a method for increasing the production of a secondary metabolite (e.g., an antibiotic, pharmacologically active compound or toxin) produced by a microorganism (e.g., a filamentous prokaryote or unicellular bacterium) by replacing a portion of the culture medium with a nutrient, such as a carbon source (e.g., glucose) or nitrogen source (e.g., an amino acid), during idiophase.

### SUMMARY OF THE INVENTION

The present invention is a method for isolating a bioactive molecule from a probiotic bacterium, e.g., from the genera Lactobacillus and Bifidobacterium. The method involves culturing pure probiotic bacteria in nutrient-rich medium for approximately 18 to 24 hours; separating the probiotic bacteria from the nutrient-rich medium; washing the probiotic bacteria with water to remove residual nutrient-rich medium; culturing the probiotic bacteria in a second medium consisting of water or water and a monosaccharide for approximately seven to 24 hours; separating the probiotic bacteria from the second medium to yield a bacteria-free supernatant containing bioactive small molecules; applying the bacteria-free supernatant to an activated porous graphitized carbon (PGC) matrix; washing the PGC matrix to remove unbound material; and eluting bound bioactive small molecules by sequential application of a polar solvent, and a polar solvent with an acidic modifier thereby isolating bioactive small molecules secreted into a bacterial culture medium.

An isolated bioactive molecule from a probiotic bacterium is also provided as are methods for using the same for activating NOD2, decreasing expression of inflammatory molecules (e.g., secretoglobins, matrix metalloproteinase-7 (MMP-7), MMP-9, osteopontin, IL-6, CXCL9, CXCL10 and CCL11), decreasing expression of gut hormones (e.g., ghrelin, gastric inhibitory peptide, insulin, leptin), inhibiting replication of human immunodeficiency virus (HIV), stimulating expression of Apolipoprotein A-IV, modulating diet-associated weight gain and preventing mucosal transmission of HIV. In addition, a pharmaceutical composition including the isolated bioactive molecule in admixture with a pharmaceutically acceptable carrier and optionally vitamin C is also provided.

### BRIEF DESCRIPTION OF THE DRAWINGS

FIG. 1 shows the amino acid content of small molecules from Lactobacillus rhamnosus GG, which were sequentially eluted from activated porous graphitized carbon (PGC) cartridges using the eluants indicated. ACN, acetonitrile; TFA, trifluoroacetic acid.

FIG. 2 is a graphical representation of positive phosphate content of small molecules recovered from different strains of Lactobacillus using activated PGC cartridges and 25% acetonitrile/0.05% TFA/water as a final phase eluant.

FIGS. 3A, 3B, 3C, 3D, 3E, 3F and 3G show liquid chromatography-mass spectrometry (LC/MS) profiles of small molecules from L. rhamnosus GG ATCC 53103 (FIG. 3A), L. jensenii ATCC 25258 (FIG. 3B), L. crispatus ATCC 33197 (FIG. 3C), L. reuteri DSM 17938(FIG. 3D), L. reuteri ATCC PTA 6475 (FIG. 3E), L. plantarum subsp. plantarum ATCC 14917 (FIG. 3F) and L. plantarum 299v DSM 9843 (FIG. 3G) analyzed in the positive ion mode. Molecules were eluted from activated PGC cartridges with 25% acetonitrile/0.05% TFA/water as a final phase eluant.

FIGS. 4A, 4B, 4C, 4D, 4E, 4F and 4G show LC/MS profiles of small molecules from L. rhamnosus GG ATCC 53103 (FIG. 4A), L. jensenii ATCC 25258 (FIG. 4B), L. crispatus ATCC 33197 (FIG. 4C), L. reuteri DSM 17938 (FIG. 4D), L. reuteri ATCC PTA 6475 (FIG. 4E), L. plantarum subsp. plantarum ATCC 14917 (FIG. 4F) and L. plantarum 299v DSM 9843 (FIG. 4G) analyzed in the negative ion mode. Molecules were eluted from activated PGC cartridges with 25% acetonitrile/0.05% TFA/water as a final phase eluant.

FIG. 5 shows the molecular mass and fragmentation patterns of bioactive small molecules (*) and structurally related products.

FIG. 6 shows the activation of NOD2 signaling by small molecules from L. rhamnosus GG ATCC 53103 sequentially eluted from PGC cartridges using the eluants indicated. ACN, acetonitrile; AA, acetic acid; FA, formic acid; TFA, triflouroacetic acid.

FIG. 7 shows the kinetics of release of small molecules with NOD2 activating activity from L. rhamnosus GG during the second culture step of the instant method. Molecules were purified at each time point using PGC cartridges and assayed for NOD2 activation. Changes in culture pH and optical density (OD₆₀₀) are also indicated.

FIGS. 8A, 8B and 8C show NOD2 and TLR2 activation associated with small molecules from different strains of Lactobacillus and Bifidobacterium. Bacteria were cultured in water (FIG. 8A), water and 1 g/L glucose (FIG. 8B), or water and 1 g/L fructose (FIG. 8C) according to the method of the invention and the crude filtered supernatants were tested for activation of NOD2 and TLR2 signaling.

FIG. 9 is a graph showing that ex vivo treatment of human full-thickness skin equivalents with a combination of small molecules from L. rhamnosus GG and Vitamin C boosts gene expression of small proline rich proteins (SPRR).

FIG. 10 shows that treatment of human ectocervical tissues with small molecules from L. rhamnosus GG (treated) as compared to untreated tissue significantly reduces secretion of osteopontin in vitro.

FIG. 11 is a graph showing that ex vivo treatment of human ectocervical tissues with small molecules from L. rhamnosus GG decreases secretion of IL-6 in donors with elevated levels of IL-6.

FIG. 12 is a graph showing that oral administration of small molecules from L. rhamnosus GG to female C57BL/6 mice results in significant increase in ApoA-IV gene expression in the small intestine within 30 minutes.

FIG. 13 shows that oral administration of small molecules from L. rhamnosus GG to female C57BL/6 mice given a high fat diet (HFD) for 8 weeks resulted in a significant increase in weight gain (Group 1) as compared to mice given a HFD and plain drinking water (Group 2). No difference in weight gain occurred in mice receiving a normal diet and either oral L. rhamnosus GG small molecules (Group 3) or plain drinking water (Group 4).

FIG. 14 shows that oral administration to C57BL/6 female mice of crude filtered supernatants of L. rhamnosus or L. reuteri cultured in either water (W) or glucose (G) according to the method of the invention for 24 weeks results in reduced weight gain in the first 12 weeks on a Western Diet (high in fat and sugar) and enhanced weight loss in the second 12 weeks on a normal diet.

### DETAILED DESCRIPTION OF THE INVENTION

Culture and fractionation methods have now been found that yield small bioactive molecules <1000 Da, in particular <500 Da, secreted by probiotic microorganisms within the genera of Lactobacillus and Bifidobacterium. The combination of culture and fractionation steps permits the separation and recovery of unique bioactive small molecules of bacterial origin. Conventionally, this process is accomplished by methods involving physical, chemical or enzymatic disruption of intact bacteria, which generally yield a poorly defined mixture or “extract” of bacterial components of variable and unknown molecular size and composition. Isolation of soluble molecules released by bacteria into culture typically requires time-consuming fractionation techniques to separate small molecules of bacterial origin from larger microbial polymers and media constituents and further require the use of expensive specialized equipment.

In contrast, this invention provides a method to isolate and recover novel bioactive small molecules of bacterial origin and involves the integration of two sequential culture steps for growing the bacteria with a third step that exploits the absorbent properties of porous graphitized carbon (PGC) to separate and recover soluble small molecules of bacterial origin that are naturally released into the culture solution or alternately combines the use of reverse-phase chromatography and size exclusion chromatography. This invention also provides for potential application of these molecules to prevent or treat conditions associated with inflammation, infection, cancer, aging, and energy homeostasis.

Accordingly, this invention is a method for isolating bioactive small molecules released by probiotic bacteria into a culture medium. In one embodiment, the method involves, in order, the steps of culturing pure probiotic bacteria (e.g., a single colony isolate) in nutrient-rich medium for approximately 18 to 24 hours; separating the bacteria from the medium (e.g., by centrifugation); washing the bacteria with water to remove residual medium; culturing the bacteria in a second medium consisting of water or water and a monosaccharide for approximately seven to 24 hours; separating the bacteria from the second medium (e.g., by centrifugation and/or filtration) to yield a bacteria-free supernatant, which contains bioactive small molecules; applying the supernatant to an activated porous graphitized carbon (PGC) matrix; washing the PGC matrix (e.g., with water) to remove unbound material; and eluting bound bioactive small molecules by sequential application of a polar solvent and a polar solvent with an acidic modifier thereby isolating bioactive small molecules released into a bacterial culture medium.

As used herein, probiotic bacteria are live microorganisms which, when administered in adequate amounts, confer a health benefit to the host. Lactic acid bacteria are the most common type of probiotic bacteria. For the purposes of the present invention, probiotic bacteria include bacteria of the genera Lactobacillus (e.g., L. rhamnosus, L. acidophilus, L. jensenii, L. plantarum, L. gasseri and L. crispatus), Streptococcus (e.g., S. thermophilus and S. salivarius), Enterococcus (e.g., E. faecalis) and Bifidobacterium (e.g., B. animalis, B. breve, B. infantis, B. lactis, and B. longum). Having demonstrated the presence of bioactive molecules in the culture medium of bacteria from each of these genera, particular embodiments of the invention embrace the use of probiotic bacteria for obtaining the bioactive molecules of the invention. In certain embodiments, the probiotic bacterium is from the genus Lactobacillus. In other embodiments, the probiotic bacterium is from the genus Bifidobacterium. In particular embodiments, the probiotic bacterium is L. rhamnosus. A pure probiotic bacterium is intended to mean that the bacterium is homogenous to one strain of bacterium. A pure probiotic bacterium can be obtained by conventional methods such as the spread plate method, the plate streaking method and/or the enrichment culture method.

A nutrient-rich medium is any medium that provides the necessary nutrients to provide robust growth of the probiotic bacterium. The nutrient-rich medium of this invention can be undefined or defined. An undefined medium is a medium that typically contains a carbon source such as glucose; water; various salts; and beef and/or yeast extract as sources of amino acids and nitrogen. By comparison, a defined medium is a medium in which all the chemicals used are known, i.e., no yeast, animal or plant tissue is presented. An example of an undefined nutrient-rich medium is DeMann, Rogosa, Sharpe (MRS) broth and derivatives thereof. A typical composition of MRS broth contains casein peptone, meat extract, yeast extract, glucose, dipotassium hydrogen phosphate, TWEEN® 80 (polysorbate 80), diammonium hydrogen citrate, sodium acetate, magnesium sulfate and manganese sulfate. Derivatives of MRS broth can include formulations containing peptones derived from fish or plant sources as a replacement for meat extract. Examples of chemically defined media include minimal growth media or cell culture media such as Dulbecco's Modified Eagle's Media (DMEM) or RPMI-1640. These media typically contain combinations of amino acids, vitamins, minerals, salts, sugars and, in some instances, pyrimidines and purines.

To provide a high yield of bacteria, the probiotic bacteria are grown at 37° C. under either anaerobic or aerobic conditions without supplemental CO₂ for approximately 18 to 24 hours (±1-2 hours). In certain embodiments, the cells are cultured for at least 18, 19, 20, 21, 22, 23 or 24 hours. In other embodiments, the cells are cultured for 18-20 hours, 19-21 hours, 20-22, 21-23, or 22-24 hours.

After growing the probiotic bacteria, the probiotic bacteria are separated from the medium. Typically, this step is carried out by centrifugation. However, other methods such as filtration and immunomagnetic separation could also be used. Once the probiotic bacteria have been separated from the nutrient-rich medium, the bacterial cells are washed to remove residual medium and hence small molecules originating from the nutrient rich medium.

Subsequently, the bacteria are cultured in a second medium consisting of water or water and a monosaccharide for approximately seven to 24 hours (±1-2 hours). In certain embodiments, the cells are cultured for at least 7, 8, 9, 10, 11, 12, 13, 14, 15, 16, 17, 18, 19, 20, 21, 22, or 24 hours. In other embodiments, the cells are cultured for 7-10 hours, 9-12 hours, 11-14, 13-16, 15-18, 17-20 or 21-24 hours. While counterintuitive, it has been found that culturing probiotic bacteria first in a nutrient-rich broth, followed by a solution of pure water or water with an added monosaccharide (e.g., glucose, fructose or galactose) results in release of microbial small molecules directly into the culture solution. It is recognized that bacteria shed cell wall components during growth, and molecular analyses indicates that the small molecules are most likely derived from the bacterial cell wall, which constitutes up to 50% of the dry weight of the bacteria. It has been observed that substitution of water with DMEM, Vegetable Peptone Broth, or other defined media for this step of the method results in unacceptably high levels of tyrosine contamination in the final products, as determined by LC/MS and amino acid analysis. Therefore, the use of water or water with an added monosaccharide is necessary for the final recovery of substantially pure products.

After the second culturing step, the bacteria are separated from the second medium or conditioned medium by, e.g., centrifugation, filtration or centrifugation and filtration, to yield a bacteria-free supernatant, which contains bioactive small molecules. To isolate the bioactive small molecules from the bacteria-free supernatant, the supernatant is applied to an activated porous graphitized carbon (PGC) matrix. Activated carbon is a known adsorbent for the purification of both gases and liquids and is recognized for its use in fractionating and de-salting oligosaccharides. However, it has now been found that PGC is an inexpensive matrix for separating and recovering complex bioactive small molecules of bacterial origin. Pre-packed PGC cartridges are commercially available (e.g., Thermo Fisher Scientific) and can be used without additional specialized equipment. To remove any material that does not bind to the matrix, the PGC matrix is washed one or more times with water.

Bioactive small molecules bound to the PGC matrix are eluted by sequential application of a polar solvent and a polar solvent with an acidic modifier. In certain embodiments, the polar solvent comprises or consists of acetonitrile in water. However, in other embodiments, acetonitrile may be used in combination or replaced with other polar aprotic solvents such as acetone, dimethylformamide, dimethyl sulfoxide or nitromethane. In certain embodiments, the bioactive small molecules are eluted by applying a 10% solution of acetonitrile (in water), and subsequently applying a 25% solution of acetonitrile, wherein eluted fractions are collected after each application of polar solvent.

Subsequent to the elution of bioactive small molecules with a polar solvent, the PGC matrix is subjected to an additional elution with a polar solvent in combination with an acidic modifier. In certain embodiments, a series of acidic modifiers are used to selectively elute bioactive small molecules. In particular embodiments, the series of acidic modifiers are sequentially added based upon increasing acidity. In certain embodiments, the acidic modifiers include acetic acid (pKₐ 4.76), formic acid (pKₐ 3.77) and trifluoroacetic acid (pKₐ 0.23). However, other acids such as propionic acid ((pKₐ 4.87), lactic acid (pKₐ 3.86), and methanesulfonic acid (pKₐ −1.9) can also be used. In particular embodiments, the polar solvent with an acidic modifier comprises or consists of sequential application of 25% acetonitrile and 0.1% acetic acid; 25% acetonitrile and 0.1% formic acid; and 25% acetonitrile and 0.05% trifluoroacetic acid, wherein eluted fractions are collected after each application of polar solvent with acidic modifier.

In accordance with one embodiment, this invention combines the use of activated PGC cartridges with two sequential culture steps: the first to encourage robust bacterial growth and the second to remove all media constituents from the culture, to yield soluble microbial small molecules <1000 Daltons, and especially <500 Daltons, that are likely shed by bacteria into the second medium and can be easily recovered in a substantially purified form. Products recovered during each of the elution steps are lyophilized and/or may be stored frozen and reconstituted in water prior to use. It has been shown that molecules recovered by this method are water soluble and suitable for downstream applications to elucidate the molecular size and composition. This includes standard amino acid analyses, combined gas chromatography/mass spectrometry (GC/MS) to determine glycan composition, determination of phosphate content and liquid chromatography-mass spectrometry (LC/MS) analyses. In addition, it has been have shown that the small molecules recovered by this method are biologically active in vitro. For example, activation of cellular Toll-like receptors associated with innate immunity, in particular NOD2 has been demonstrated.

In an alternative embodiment, bioactive molecules of the present invention are isolated by passing conditioned medium through a reverse-phase chromatography matrix to obtain hydrophilic molecules having a molecular weight of less than 1000 Daltons; and further fractionating the flow-through from the reverse-phase chromatography matrix by size exclusion. Size exclusion fractions embraced by the present invention contain bioactive molecules characterized as being hydrophilic; stable to at least 99° C.; stable at pH from about 3.0 to about 10.0; resistant to protease digestion; and as having a molecular weight of between about 500 Daltons and about 700 Daltons. In addition to these physical characteristics, the bioactive molecules of the invention have a variety of biological activities.

Reverse-phase chromatography includes any chromatographic method that uses a non-polar stationary phase. Samples applied to a reverse-phase chromatography matrix are separated based on the principle of partitioning between the mobile and stationary liquid phases and can be carried out by means of either a high-performance liquid chromatography column or a fast protein liquid chromatography column. In general, the mobile phase encompasses two solvent solutions, a polar and a non-polar solvent, to be blended via a gradient over the course of the chromatographic separation. Upon application of probiotic bacterium-conditioned media to a reverse-phase chromatography matrix, the initial flow-through is collected. This flow-through contains salts, hydrophilic amino acids and small hydrophilic peptides of less than 1000 Daltons. It is in this initial flow-through that the bioactive molecules of the present invention are found. Reverse-phase matrices suitable for purification of bioactive molecules of this invention include columns packed with silica beads bearing alkyl groups ranging in length from 4-18 carbon atoms, i.e., C4-C18. In particular embodiments, the reverse-phase chromatography matrix is a C18 matrix. Reagents and methods for carrying our reverse-phase chromatography are routinely practiced in the art. See, e.g., Scopes, et al. (January 1994) In: Protein Purification: Principles and Practice, 3rd edition, Springer Verlag.

Flow-through collected from the reverse-phase chromatography matrix is subsequently applied to a size exclusion column to elute and isolate fractions containing hydrophilic bioactive molecules of 500 Dalton to 700 Dalton in size. Any suitable size exclusion method can be used including, e.g., gel filtration chromatography, gel permeation chromatography, or gel electrophoresis. The matrix for size exclusion can be polyacrylamide, dextran, agarose, silica or crosslinked polystyrene. Commonly employed size exclusion matrices include SUPERDEX-, SEPHADEX-, SUPEROSE-, SEPHAROSE- and SEPHACRYL-based matrices. Fractions containing the bioactive molecules of the invention can be identified based upon the presence of molecules having one or more of the biophysical characteristics or biological activities of the bioactive molecules of the invention. Analysis of fractions can be carried out as described herein or using any other conventional method for determining such biophysical characteristics or biological activities.

By combining particular culturing practices and reverse-phase chromatography/size exclusion methods, small bioactive molecules (500-700 Da) secreted by the lactic acid bacterium Lactobacillus rhamnosus GG (LGG) have been obtained, which are hydrophilic, heat stable, acid stable, and resistant to proteolytic enzymes. Furthermore, the molecules decrease HIV replication in susceptible target cells and tissues, decrease secretion of inflammatory immune mediators (e.g., IL-1ra, IL-6, and IL-8), decrease secretion of vasoendothelial growth factor (VEGF), and decrease activation of cell signaling through MAPK (Erk1/Erk2; extracellular signal-regulated kinases 1/2) pathways. Given their potent activity, these molecules find application as topical microbicides for preventing HIV transmission in both adults and infants. For example, the bioactive molecules herein can be formulated in a topical microbicide for vaginal or rectal use to prevent HIV sexual transmission or as a topical or oral formulation for use in infants exposed to HIV through breastfeeding.

It is contemplated that the bioactive molecules of the invention can be used as a fraction obtained from the PGC or size exclusion matrix or alternatively be further purified to homogeneity. Methods for purifying one or more bioactive molecules to homogeneity (e.g., greater than 90%, 95%, or 99% purity) include thin layer chromatography, SDS-PAGE, ion exchange HPLC, and the like. Such methods are routinely employed in the art to purify molecules of interest and any one or combination of methods can be employed.

The bioactive molecules of the invention are isolated in the sense that the bioactive molecules have been removed from their natural environment, i.e., conditioned medium, in a form to achieve a significant increase in activity over crude extracts having said bioactive molecules. Such isolated bioactive molecules can include, but are not limited to, bioactive molecules purified to homogeneity, recombinantly produced bioactive molecules, and isolated bioactive molecules which have been fractionated by column chromatography (e.g., PGC or reverse-phase/size exclusion).

The bioactive molecules of the present invention can include, but are not limited to, small organic molecules, e.g., small peptides or oligosaccharides that are heat and acid stable, and demonstrate significant ability to modulate cellular inflammatory responses and/or inhibit HIV replication. In so far as the bioactive molecules are stable and biologically active under a variety of conditions, these molecules are particularly useful, alone or in pharmaceutical compositions, for activating NOD2, decreasing the expression of inflammatory molecules (e.g., secretoglobins, matrix metalloproteinase-7 (MMP-7), MMP-9, osteopontin, IL-6, CXCL9, CXCL10 and CCL11), decreasing expression of gut hormones that regulate energy metabolism (e.g., ghrelin, gastric inhibitory peptide, insulin, leptin), increasing the expression of SPRR, for inhibiting HIV replication, stimulating expression of Apolipoprotein A-IV, modulating weight gain and preventing mucosal transmission of HIV in vitro or in vivo.

Accordingly, the present invention further provides the use of the bioactive molecules of the invention for activating NOD2, increasing the expression of SPRR, decreasing the expression of genes involved in inflammation and reproductive tract cancers (e.g., osteopontin, secretoglobins, WFDC2/HE4), decreasing expression of gut hormones that regulate energy metabolism (e.g., ghrelin, gastric inhibitory peptide, insulin, leptin), inhibiting HIV replication, decreasing expression of inflammatory cytokines and chemokines, decreasing expression of VEGF and decreasing activation of Erk1/Erk2 in a cell. Cells of particular application in accordance with such methods include human epithelial cells and cells of primary tissues which contain epithelial cells and CD4+ cells. Such cells are of particular interest in so far as they are located at mucosal surfaces and are in direct contact with or susceptible to infection by pathogens such as HIV. In this regard, some embodiments of the invention provide for cells already infected with HIV, as well as cells that are free of HIV. In carrying out these methods, a cell is contacted with an effective amount of one or more isolated bioactive molecules of the invention such that a detectable decrease in HIV replication (e.g., as determined by transcription of HIV), inflammatory cytokine and chemokine expression (e.g., IL-1ra, IL-6 or IL-8), VEGF expression, or Erk1/Erk2 activation is achieved as compared to a cell not contacted with the isolated bioactive molecules. A detectable decrease includes at least a 20%, 30%, 40%, 50%, 60%, 70%, 80%, 90%, or 100% decrease as determined using the methods disclosed here or any other suitable methods routinely practiced in the art for detecting HIV replication, inflammatory cytokine and chemokine expression, VEGF expression, or Erk1/Erk2 activation.

In so far as VEGF, Erk1/Erk2, and proinflammatory cytokine and chemokines such as IL-6, IL-1ra, and IL-8 are involved in a variety of diseases and conditions besides HIV infection, the disclosed methods of the invention also find application in the prevention and treatment of such diseases and conditions. For example, increased IL-8 expression is associated with poor clinical outcome in human ovarian carcinoma, and decreasing IL-8 expression has been shown to decrease tumor growth through antiangiogenic mechanisms (Merritt, et al. (2008) J. Natl. Cancer Inst. 100(5):359-72). Similarly, inhibition of VEGF expression has been shown to inhibit the growth of colorectal cancer. (Lv, et al. (2007) Cancer Biother. Radiopharm. 22(6):841-52). In addition, inhibition of the Erk pathway has been shown to prevent HIVgp120-induced REM sleep increase (Diaz-Ruiz, et al. (2001) Brain Res. 913:78-81). Accordingly, the bioactive molecules of the invention can be used in the treatment of diseases and conditions including, but not limited to, autoimmune diseases, cancer, infections, as well as tissue damage due to inflammatory responses.

While a direct interaction with HIV was not identified, the bioactive molecules of the invention were shown to decrease activation of host cell pathways, which regulate HIV transcription and therefore HIV replication and transmission. Accordingly, the present invention further embraces a method for preventing mucosal transmission of HIV by administering to the mucosa of a subject an effective amount of the bioactive molecules of the invention, or a pharmaceutical composition containing such bioactive molecules. In particular embodiments, the bioactive molecules are topically applied in an acid-buffering gel or cream to prevent mucosal transmission of HIV-1 in the female reproductive tract, or with a neutral pH gel or cream to prevent rectal transmission of HIV-1 in adults.

Bioactive molecules of the present invention can be conveniently used or administered in a composition containing one or more active molecules in combination with a pharmaceutically acceptable carrier. Such compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A carrier, pharmaceutically acceptable carrier, or vehicle, such as a liquid or solid filler, diluent, excipient, or solvent encapsulating material, is involved in carrying or transporting the subject active molecules from one organ, or portion of the body, to another organ, or portion of the body. Each carrier must be acceptable in the sense of being compatible with the other ingredients of the formulation and not injurious to the patient.

Examples of materials which can serve as carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Bioactive molecules of the invention can be administered via any route including, but not limited to, oral, rectal, buccal (e.g., sub-lingual), vaginal, parenteral (e.g., subcutaneous, intramuscular, intradermal, intravenous, intraperitoneal), topical (i.e., both skin and mucosal surfaces, including vaginal and gastrointestinal surfaces), intranasal, transdermal, intraarticular, intrathecal and inhalation administration. The most suitable route in any given case will depend on the nature and severity of the condition being prevented or treated and on the nature of the particular bioactive molecule(s) being used.

For injection, the carrier will typically be a liquid, such as sterile pyrogen-free water, pyrogen-free phosphate-buffered saline solution, bacteriostatic water, or CREMOPHOR (Polyoxyl 35 Castor Oil; BASF, Parsippany, N.J.). For other methods of administration, the carrier can be either solid or liquid.

For oral administration, the compound can be combined with one or more carriers and used in the form of ingestible tablets, buccal tablets, troches, capsules, elixirs, suspensions, syrups, wafers, chewing gums, foods and the like. Such compositions and preparations typically contain at least 0.1% of bioactive molecule(s). The percentage of the bioactive molecule (s) and preparations can, of course, be varied and can conveniently be between about 0.1 to about 100% of the weight of a given unit dosage form. The amount of bioactive molecule(s) in such compositions is such that an effective dosage level will be obtained. In particular embodiments, the bioactive molecules are formulated in an oral composition for use in preventing HIV-1 transmission in the gastrointestinal tract of infants exposed to HIV-1 through breastfeeding. The isolated bioactive molecules can be combined with a neutral pH agent (e.g., a gel, cream or infant formula) for oral application.

When prepared in the form of tablets, troches, pills, capsules, and the like, such formulations can also contain binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

A syrup or elixir can contain the active agent, sucrose or fructose as a sweetening agent, methyl and propylparabens as preservatives, a dye and flavoring such as cherry or orange flavor. Of course, any material used in preparing any unit dosage form should be substantially non-toxic in the amounts employed. In addition, the active compounds can be incorporated into sustained-release preparations and devices including, but not limited to, those relying on osmotic pressures to obtain a desired release profile.

Formulations of the present invention suitable for parenteral administration contain sterile aqueous and non-aqueous injection solutions of the bioactive molecules, which preparations are generally isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3(6):318 (1986)) and typically take the form of an optionally buffered aqueous solution of the compound. Suitable formulations contain citrate or Bis\Tris buffer (pH 6) or ethanol/water and contain from 0.1 to 0.2 M of the compound.

A compound can alternatively be formulated for nasal administration or otherwise administered to the lungs of a subject by any suitable means. In particular embodiments, the compound is administered by an aerosol suspension of respirable particles containing the bioactive molecule(s), which the subject inhales. The respirable particles can be liquid or solid. The term aerosol includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn, et al. (1992) J. Pharmacol. Toxicol. Methods 27:143-159. Aerosols of liquid particles containing the bioactive molecule(s) can be produced by any suitable means, such as with a pressure-driven aerosol nebulizer or an ultrasonic nebulizer, as is known to those of skill in the art. See, e.g., U.S. Pat. No. 4,501,729. Aerosols of solid particles containing the bioactive molecule(s) can likewise be produced with any solid particulate medicament aerosol generator, by techniques known in the pharmaceutical art.

Formulations suitable for topical application to the skin can take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof. In particular, bioactive molecules of the invention can be formulated with specific acid-buffering agents, in the form of gels or creams, designed for topical use. Acid-buffering products that maintain vaginal fluid at a mildly acidic pH (4.5-5.0) are conventionally employed in clinical trials as topical microbicides to prevent HIV-1 infection (e.g., BUFFERGEL™; ReProtect Inc., Baltimore, Md.). Accordingly, particular embodiments of the invention embrace a pharmaceutical composition containing the isolated bioactive small molecules with an acid-buffering gel or cream for preventing HIV-1 infection across mucosal surfaces in the vagina. In alternative embodiments, formulation of the bioactive molecules with a neutral pH gel or cream can be used for rectal use against HIV-1 sexual transmission.

In particular embodiments, the bioactive molecules of the invention are administered to a subject in an effective amount. Dosages of bioactive molecules can be determined by methods known in the art, see, e.g., Remington: The Science and Practice of Pharmacy, supra. The selected effective dosage level will depend upon a variety of factors including the activity of the particular bioactive molecule(s) employed, the route of administration, the time of administration, the rate of excretion or metabolism of the particular bioactive molecule(s) being employed, the duration of the treatment, other drugs, compounds and/or materials used in combination with the particular bioactive molecule(s) employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required for prevention or treatment in a subject (e.g., a human) based upon clinical and preclinical trials of the pharmaceutical composition for the disease or condition being prevented or treated.

Such preclinical trials can include the evaluation of toxicity and cytopathology following topical application of the bioactive molecules directly to vaginal tissues in a murine model. In addition, preclinical data can be obtained regarding the impact of body fluids, namely vaginal secretions and human serum, on the bioactivity of the isolated bioactive molecules disclosed herein.

The invention is described in greater detail by the following non-limiting examples.

### Example 1

### Isolation of Bioactive Small Molecules from Defined Media

Bacterial Culture in Undefined and Defined Media:

The Lactobacillus rhamnosus GG (LGG) strain was obtained from ATCC (Manassas, Va.) under Accession number 53103. LGG bacteria were inoculated from a glycerol stock into MRS broth and cultured overnight under aerobic conditions at 37° C. without supplemental CO₂. The bacteria were centrifuged and washed twice with serum-free Dulbecco's Modified Eagle's Media (DMEM, neutral pH 7.1) to remove all MRS broth from the culture. The washed bacteria were inoculated into fresh serum-free DMEM at neutral pH 7.0-7.1 at a density of 0.5 OD₆₀₀, and cultured for an additional eight to 24 hours under aerobic conditions at 37° C. without supplemental CO₂. The culture was centrifuged to pellet the bacteria. The conditioned culture supernatant was separated from the bacterial pellet and was adjusted to neutral pH 7.1 with NaOH. Conditioned supernatant from L. rhamnosus GG cultures as well as other Lactobacilli including L. acidophilus (ATCC 4356), L. jensenii (ATCC 25258), L. plantarum (ATCC 14917), L. gasseri (ATCC 9857) and L. crispatus (ATCC 33197), and bacteria within the genera of Bifidobacterium, Streptococcus and Enterococcus (e.g., E. faecalis, ATCC 700802) indicate that these bacteria produce soluble factors that reduce HIV-1 replication in vitro.

Reverse Phase HPLC/C18 Fractionation.

Conditioned supernatants from L. rhamnosus GG cultures were evaporated to dryness in a SPEED-VAC concentrator under dry ice. The dried pellet was resuspended in a small volume of H₂O/0.1% TFA and loaded onto a C18 reverse-phase column. The initial flow-through fraction was collected, while all other compounds retained on the column were removed. The flow-through fraction contained salts, hydrophilic amino acids and hydrophilic small peptides (<1000 Da)

SEPHADEX G-10 Fractionation.

The flow-through fraction of the reverse phase column was de-salted and the low molecular weight compounds (<300 Da) were removed by size exclusion chromatography on a SEPHADEX G-10 column.

Mass Spectrometry and Edman's Sequencing.

G-10 fractions were analyzed by Mass Spectrometry using MALDI-TOF and demonstrated a pattern of distinct peaks in the range of 500 Da to 700 Da. Edman's sequencing demonstrated a pattern of hydrophilic amino acids consistent with small peptides of four to six amino acid residues in length. Hydrophilic amino acids identified included, Aspartic Acid (D), Glutamic Acid (E), Arginine (R), Hisitidine (H), Serine (S), Threonine (T) and Glutamine (Q).

### Example 2

### Characterization of Isolated Bioactive Molecules from Defined Media

Defined media fractions containing bioactive small molecules were screened for activity on human cells of vaginal, cervical, intestinal, mammary, and lymphoid origin. Specifically, bioactivity and HIV-inhibition were evaluated in one or more of the following human cells, cell lines and tissues: CD4+ cells (TZM-bl), primary CD4+ lymphocytes, primary cervicovaginal tissues, mammary epithelial cells (MFC-10A), and intestinal epithelial cells (Caco-2).

Inhibition of HIV Replication.

TZM-bl cells are genetically engineered human cells that are highly susceptible to HIV-1 infection. These cells were treated with individual SEPHADEX G-10 fractions and infected with HIV-1. HIV inhibition was determined relative to media controls, and active fractions containing HIV-inhibitory activity were identified. The active fractions were evaluated by MALDI-TOF and demonstrated the presence of small molecules in the range of 500-700 Da.

Mechanism of Action Against HIV.

HIV inhibition was evaluated by culture and PCR methods to determine whether the bioactive molecules block the early stages of the virus life cycle. Infection and replication of HIV-1 in activated CD4+T lymphocytes, the primary target cell for HIV-1 in vivo, was evaluated. No significant effect was observed for HIV binding, entry, reverse transcription or integration when exposed to the bioactive molecules. Treatment was not associated with inhibition of viral binding to the target cell (absorption) via the envelope glycoproteins. Stimulation of integrated HIV-1 through the virus-long-terminal repeat (LTR) region indicated a reduction in expression at the level of virus transcription (post-integration). This finding does not preclude additional effects on later stages of the virus life cycle. Taken together, these results indicate that the bioactive small molecules produced by probiotic bacteria act to reduce expression of HIV-1 once the virus is integrated into the host cell, most likely through modulation of host cell pathways involved in regulating viral transcription.

Effect on Human Immune Mediators.

The effect of bioactive small molecules was tested in human cells and tissues for the ability to modulate expression of host immune mediators. Cultures of human cells or primary tissue explants were treated for 48 hours with SEPHADEX G-10 fractions containing bioactive small molecules (500-700 Da), or appropriate controls, and the culture supernatants were tested in multiplex LUMINEX assays against a panel of different human cytokines, chemokines and growth factors. Modulation in expression of human cytokines and chemokines, including, for example, IL-1ra, IL-6, IL-8, MCP-1, and IP-10, was noted for treated cervicovaginal tissues, and epithelial cells of mammary origin (MFC-10A). Modulation of cytokine and chemokine expression in human intestinal epithelial cells (Caco-2 cells) and primary human lymphocytes was also conducted to demonstrate the effects of the small bioactive molecules on these cell types. SEPHADEX G-10 fractions that decreased expression of immune mediators also decreased HIV-1 infection in parallel assays, suggesting a common mechanism of action involving host cell pathways that regulate both HIV transcription and expression of immune mediators.

Decreased Expression of Human Vasoendothelial Growth Factor (VEGF).

LUMINEX assays revealed a decrease in expression of human VEGF in primary human cervicovaginal tissues treated with SEPHADEX G-10 fractions containing small bioactive molecules of 500-700 Da. The levels of VEGF secreted into the culture supernatants were significantly reduced (≧10-fold) in some of the treated tissues as compared to untreated controls. Similar results were obtained using human mammary epithelial cells (MFC-10A). The same SEPHADEX G-10 fractions that decreased VEGF expression also decreased HIV-1 replication, and down-regulated immune mediators (e.g., IL-1ra, IL-6, IL-8), thereby suggesting a common cellular signaling pathway involved in regulation of both HIV transcription and expression of host growth factors such as VEGF.

Decreased Activation of Cellular MAPK (Erk1/2) Pathways.

In further experiments, CD4+ TZM-bl cells were stimulated with serum to activate cellular MAPK pathways, either in the presence or absence of SEPHADEX G-10 fractions containing small bioactive molecules of 500-700 Da. Phosphorylation of Erk1/2 was detected in cell lysates by western blot analysis. Decreased Erk1/2 phosphorylation was demonstrated as early as five minutes after serum stimulation in cells treated with fractions containing small bioactive molecules of 500-700 Da as compared to control cells treated with media alone. These results indicate that the bioactive small molecules disclosed herein may decrease both HIV replication and host cell activation by inhibiting induction of this kinase pathway. Thus, the bioactive molecules do not appear to significantly interact with the virus itself, but rather act on the target cell to limit virus replication.

Biophysical Properties.

Small bioactive molecules isolated from the probiotic bacteria disclosed herein were treated with either heat (99° C. for 15 minutes), protease digestion (trypsin, protease each at 100 μg/ml), or adjusted to pH 3.0-10.0 and then back to neutral pH, and were found to retain biological activity against HIV-1 under each of these conditions. Sensitivity to additional proteolytic enzymes including Proteinase K and pronase will indicate whether the small bioactive molecules are generally resistant to all proteases.

### Example 3

### Two Step Culturing Method with Nutrient-Rich Medium and Water

Effect of Initial Culture Medium on Growth Kinetics.

Lactobacillus rhamnosus GG (ATCC 53103; CULTURELLE) inoculated directly from a pure glycerol stock was cultured for 18 hours in MRS broth, Vegetable Peptone Broth (VPB), DMEM, or water with 4.5 g/l glucose and density was monitored hourly at OD₆₀₀. This analysis indicated that while water/glucose and DMEM did not support the initial growth phase of L. rhamnosus GG (OD₆₀₀ ˜0), VPB and MRS increased density to ˜0.75 and 2.0 OD₆₀₀, respectively. Therefore, culture of a pure strain of bacteria in an undefined nutrient rich media for at least 18 hours achieves robust growth of the bacteria.

Growth of an Established Culture in Different Culture Media.

To assess whether there is a change in growth kinetics based upon inoculum density, L. rhamnosus GG, at a starting density of 0.5 OD₆₀₀, was inoculated into MRS broth, VPB, DMEM or water with 4.5 g/l glucose and density was monitored every two hours at OD₆₀₀. After eight hours, the density of L. rhamnosus GG in water/glucose marginally increased to ˜0.6, whereas growth in DMEM, VPB and MRS increased the density to ˜0.9, 1.0 and 2.0 OD₆₀₀, respectively.

In light of these results, a two step culturing method was developed wherein a pure culture is inoculated into nutrient-rich medium and grown for 18-24 hours, and the cells are collected by centrifugation and subsequently cultured in water±monosaccharide for 7-24 hours.

### Example 4

### In Vitro Characterization of Bioactive Small Molecules from Water as Medium

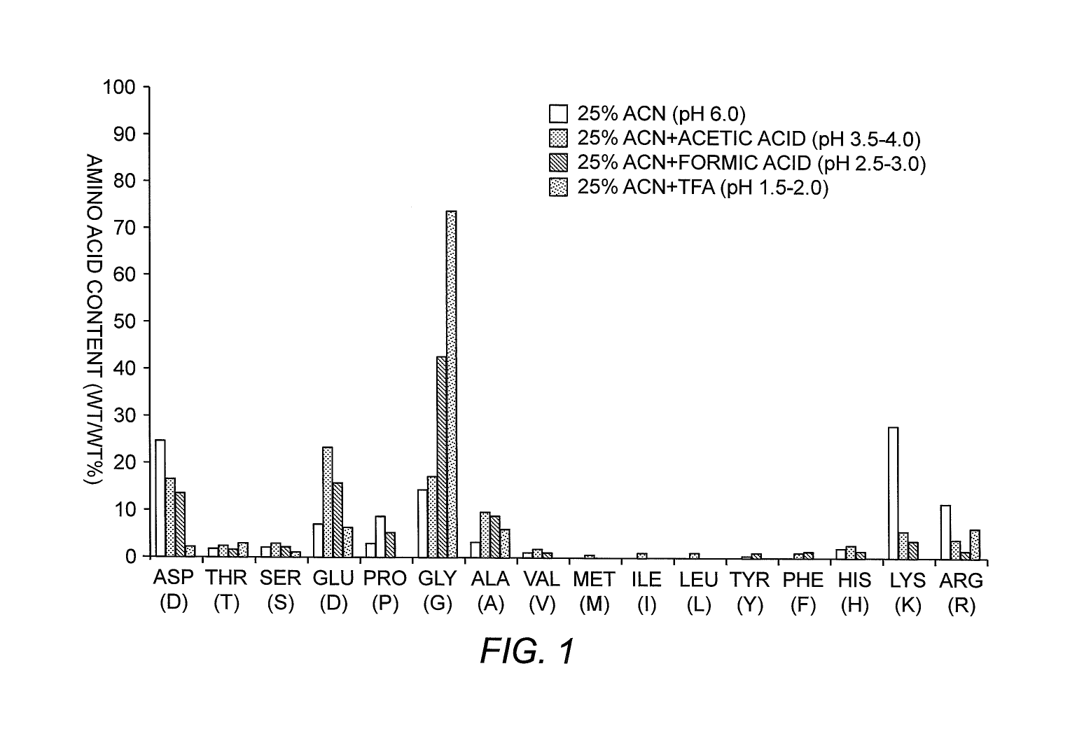

Lactobacillus rhamnosus GG was cultured as described in Example 3. Bacteria-free, conditioned supernatant was filtered and passed through an activated PGC cartridge. The cartridge was washed extensively with water to remove unbound material. Bound material, representing bacterial components, was eluted by sequential application of 10% acetonitrile/water (pH 6.0), 25% acetonitrile/water, 25% acetonitrile/0.1% acetic acid/water (pH 3.5-4.0), 25% acetonitrile/0.1% formic acid/water (pH 2.5-3.0) and 25% acetonitrile/0.05% TFA/water (pH 1.5-2.0). Products recovered during each of the elution steps were subjected to standard amino acid analyses, which demonstrated that each fraction contained different products (FIG. 1). Likewise, when similar analysis was carried out for culture supernatants of L. reuteri (DSM 17938), L. reuteri (ATCC PTA 6475), L. plantarum subsp. plantarum (ATCC 14917), L. plantarum 299v (DSM 9843), L. crispatus (ATCC 33197) and L. jensenii (ATCC 25258), each fraction for each bacterium contained a different amino acid composition. Moreover, the amino acid contents of closely related strains of the same species were distinct for each fraction.

Combined gas chromatography/mass spectrometry (GC/MS) analysis was also conducted to determine glycan composition. The glycosyl composition of products eluting from PGC matrix is presented in Table 1.

TABLE 1

| | |
|---|---|
| | Mass, mg |
| Glycosyl | (Mol %)¹ |

| Residue | F1 | F2 | F3 | F4 |
|---|---|---|---|---|
| Ribose | 199.0 | 78.7 | 44.0 | 21.7 |
| | (55.7) | (97.8) | (97.9) | (98.8) |
| Arabinose | n.d. | n.d. | n.d. | n.d. |
| Rhamnose | n.d. | n.d. | n.d. | n.d. |
| Fucose | n.d. | n.d. | n.d. | n.d. |
| Xylose | n.d. | n.d. | n.d. | n.d. |
| Glucuronic | n.d. | n.d. | n.d. | n.d. |
| Acid | | | | |
| Galacturonic | n.d. | n.d. | n.d. | n.d. |
| Acid | | | | |
| Mannose | n.d. | n.d. | n.d. | n.d. |
| Galactose | 0.7 | n.d. | n.d. | n.d. |
| | (0.2) | | | |
| Glucose | 189.4 | 2.1 | 1.1 | 0.3 |
| | (44.1) | (2.2) | (2.1) | (1.20 |
| GalNAc | n.d. | n.d. | n.d. | n.d. |
| GlcNAc | n.d. | n.d. | n.d. | n.d. |
| ManNAc | n.d. | n.d. | n.d. | n.d. |
| Total | 389.0 | 80.8 | 45.1 | 22.0 |

F1, 25% acetonitrile/water. F2, 25% acetonitrile/0.1% acetic acid/water. F3, 25% acetonitrile/0.1% formic acid/water. F4, 25% acetonitrile/0.05% TFA/water. GalNAc, N-Acetyl Galactosamine. GlcNAc, N-Acetyl Glucosamine. ManNAc, N-Acetyl Mannosamine. n.d., not detectable.
¹Values are expressed as mole percent of total carbohydrate.

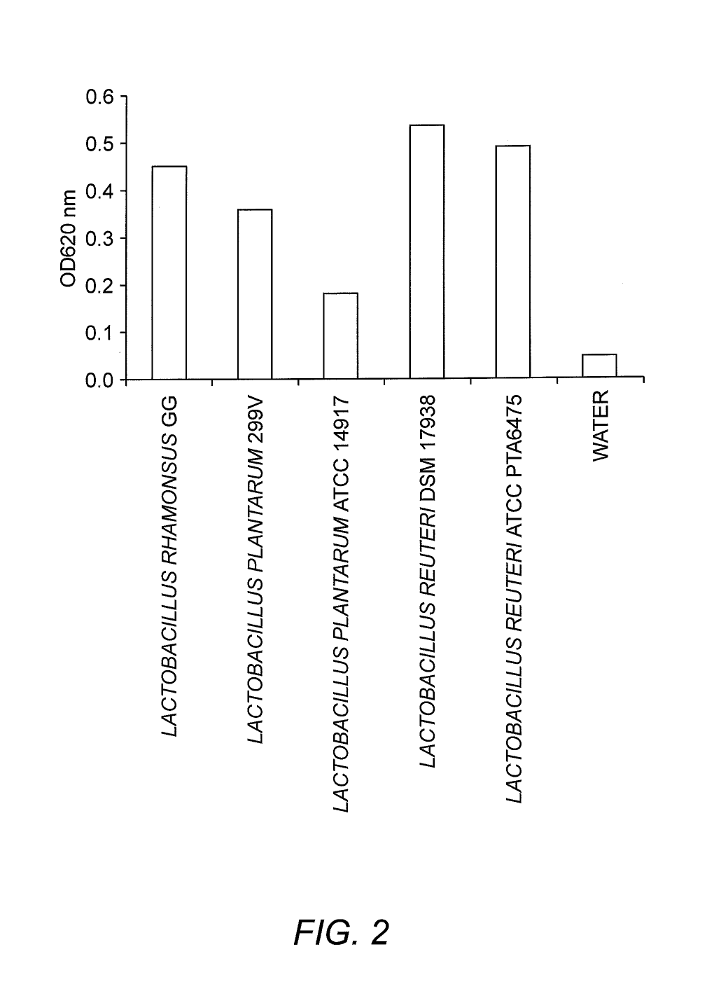

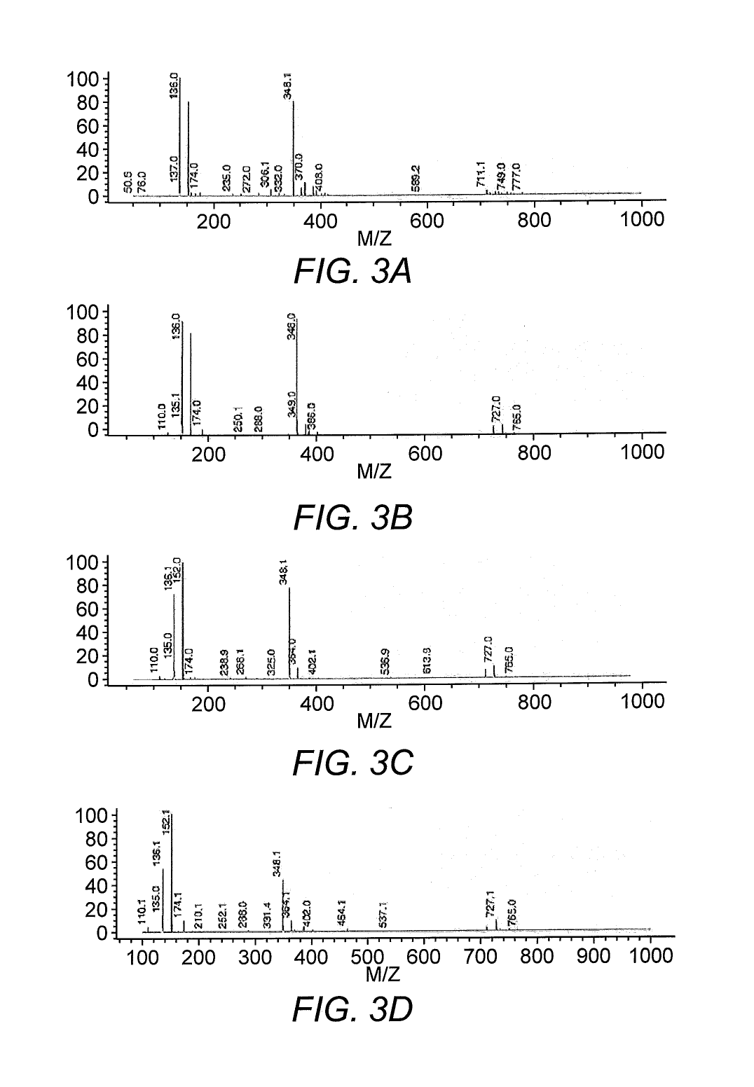

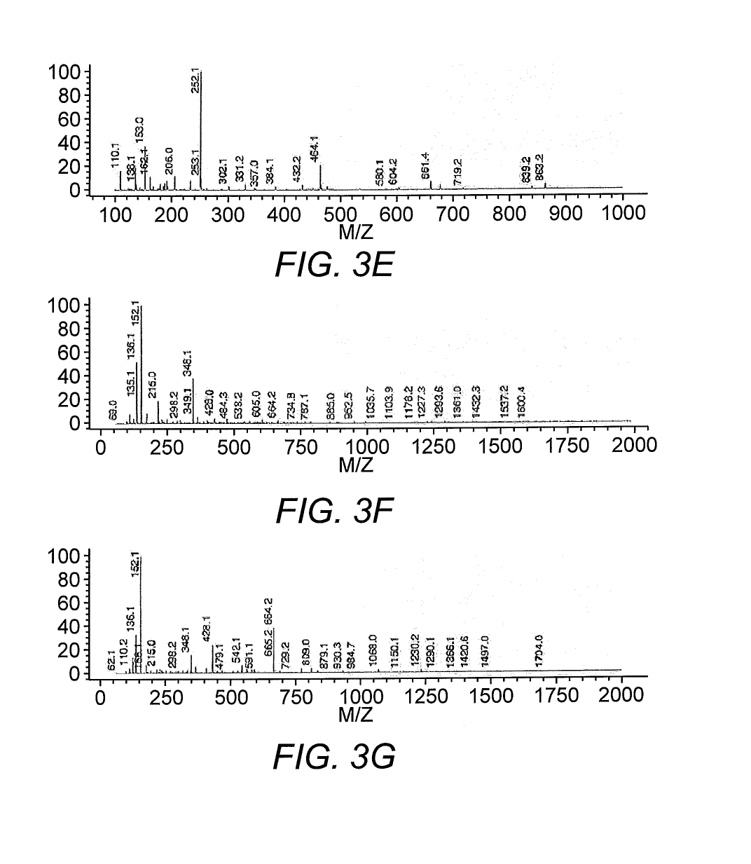

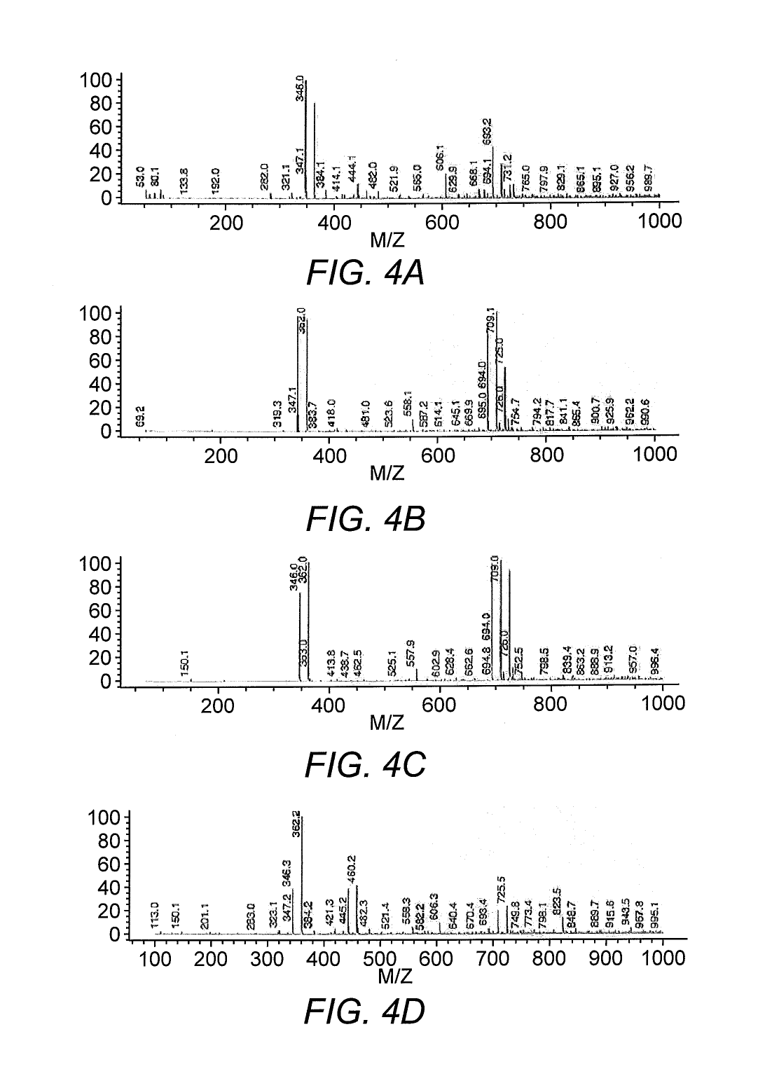

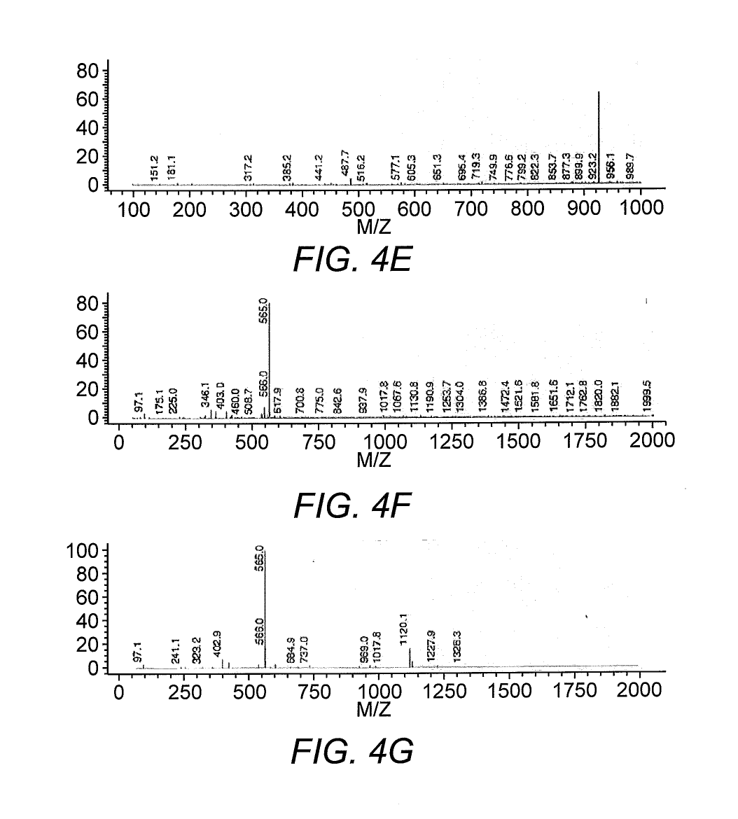

The phosphate content of small molecules recovered from different strains of Lactobacillus using activated PGC cartridges was also compared. This analysis indicated that molecules present in the final elution step (25% acetonitrile/0.05% TFA/water) for each bacterium were different (FIG. 2). Liquid chromatography-mass spectrometry (LC/MS) analyses of these same fractions in positive (FIGS. 3A-3G) and negative (FIGS. 4A-4G) ion modes revealed the presence of different molecules amongst these strains.

The molecular mass and fragmentation patterns of bioactive molecules and structurally related products were also determined. Crude material (150 ml) from pure cultures of bacteria were prepared according to the two-step culture method and applied to activated PGC cartridges as described herein. Bound products were sequentially eluted with 25% acetonitrile/water, 25% acetonitrile/0.1% acetic acid/water, 25% acetonitrile/0.1% formic acid/water and 25% acetonitrile/0.05% trifluoroacetic acid/water in a volume of 4 ml each. Eluted material was lyophilized and resuspended in 100 μl of water and analyzed by HPLC using a DIONEX Ultimate 3000 system with a HYPERSIL GOLD C18 column (50×2.1 mm 1.9 um) and a C18 guard column (10×2.1) kept at 40° C.

Gradient separation used 5-100% acetonitrile+0.1% formic acid over 15 minutes, held at 100% acetonitrile+0.1% formic acid for 2 minutes before returning to starting conditions and re-equilibrating for 5 minutes. A Flow rate of 0.3 mL/minute was used. Samples were stored at 8° C. in an autosampler prior to injection. Full scan analysis injected 10 μl/sample; product scan analysis injected 5 μl/sample.

MS Detection was performed using a TSQ Vantage triple quadrupole mass spectrometer under the following parameters: Full scan analysis to detect unknown compounds used a positive ion ESI MS scanning 100-1500 m/z in Q1. The product scan analysis to detect products from each compound of interest used positive ion ESI MS/MS scanning in Q1, Collision Energy 20-30 Volts in Q2, and scanning 50−X m/z in Q3. Generic ESI settings were in place: Spray Voltage 4000, Vaporizer Temp 350° C., Capillary Temp 380° C., Sheath Gas and Aux Gas pressures at 35 and 5, respectively.

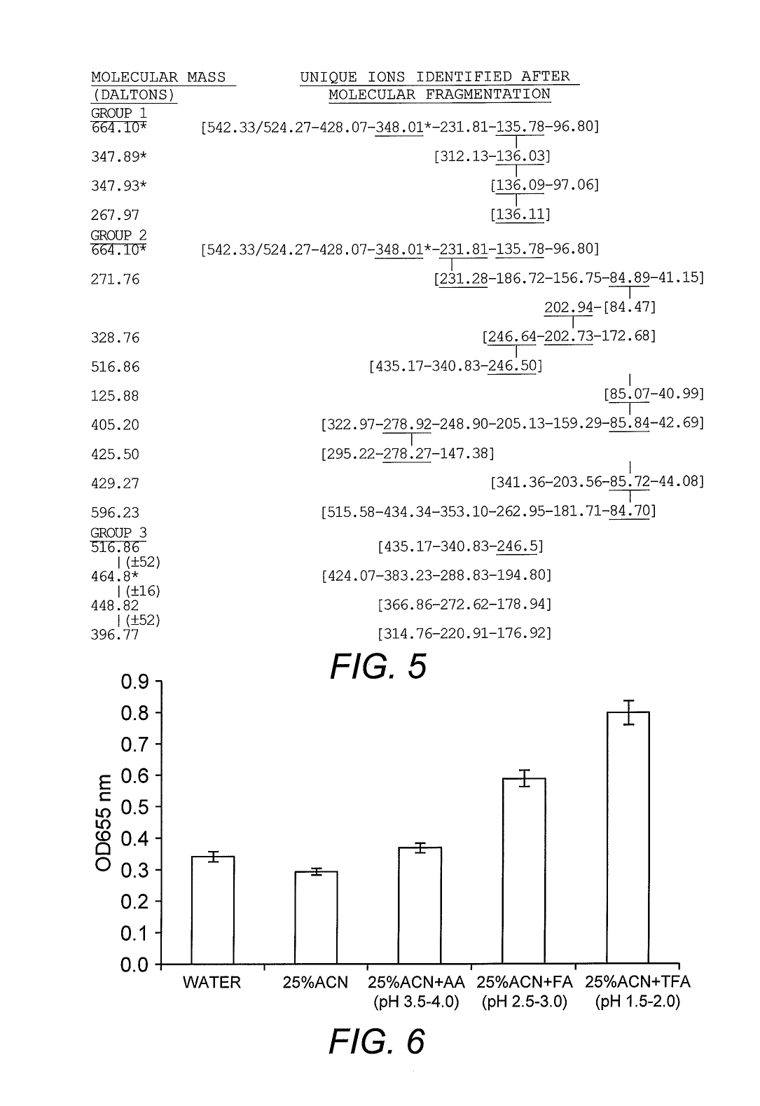

Unique abundant molecular ions were identified in eluted products by comparison to a similar analysis of water. Fragmentation patterns for parent ions were determined and related patterns were aligned and grouped. FIG. 5 shows the unique abundant parent ions and fragmentation patterns derived from products with biologic activity, including products with related ion fragmentation patterns. Table 2 shows the source of unique abundant ions presented in FIG. 5.

TABLE 2

| | | |
|---|---|---|
| Sample | Fraction | parent m/z |

GROUP 1

| | | |
|---|---|---|
| L. rhamnosus | TFA | 664.10 |
| L. reuteri | AA | 347.89 |
| L. rhamnosus | TFA | 347.93 |
| L. rhamnosus | FA | 267.97 |

GROUP 2

| | | |
|---|---|---|
| L. rhamnosus | TFA | 664.10 |
| L. reuteri | TFA | 271.76 |
| L. reuteri | TFA | 328.76 |
| L. reuteri | TFA | 516.86 |
| L. reuteri | TFA | 125.88 |
| L. rhamnosus | FA | 405.20 |
| L. reuteri | ACN | 429.27 |
| L. reuteri | ACN | 596.23 |

GROUP 3

| | | |
|---|---|---|
| L. reuteri | TFA | 516.86 |
| L. reuteri | TFA | 464.8 |
| L. reuteri | TFA | 448.82 |
| L. reuteri | TFA | 396.77 |

TFA = 25% acetonitrile/0.05% trifluoroacetic acid/water.
FA = 25% acetonitrile/0.1% formic acid/water.
AA = 25% acetonitrile/0.1% acetic acid/water.
ACN = 25% acetonitrile/water.

The biological activity of the recovered small molecules was also analyzed. Specifically, it was found that small molecules from L. rhamnosus GG could activate cellular Toll-like receptors associated with innate immunity, in particular NOD2 (FIG. 6). NOD2 activating molecules were also present in other strains of Lactobacillus.

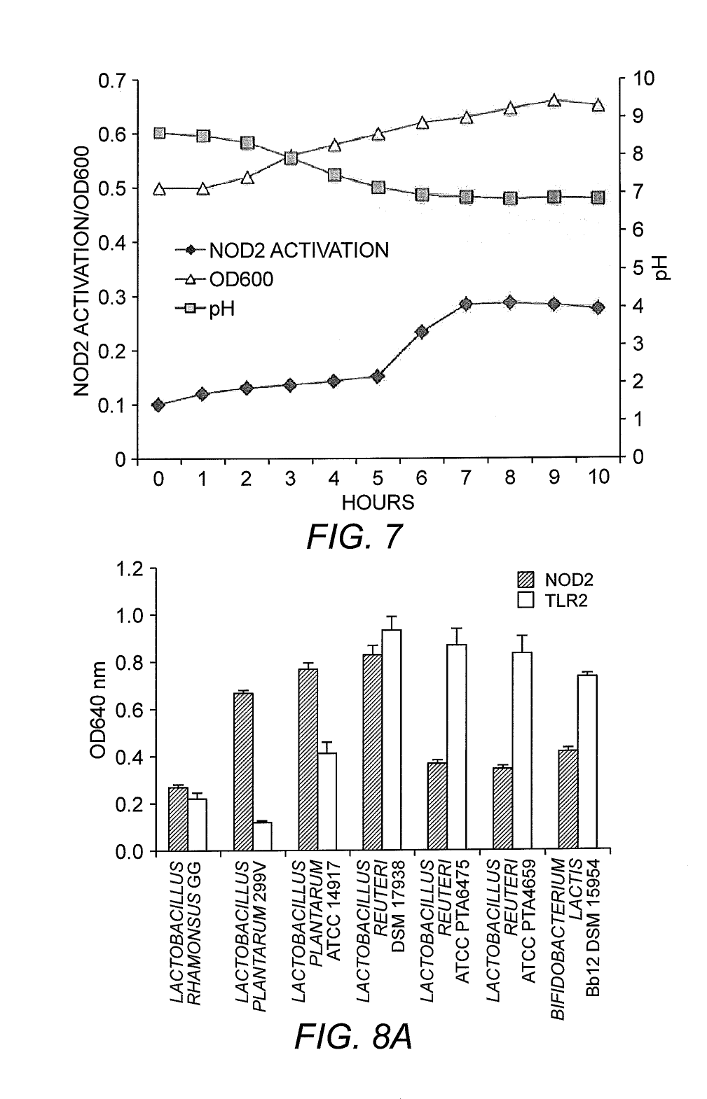

To determine the kinetics of release of the NOD2-activating activity into the culture supernatant, L. rhamnosus GG was cultured in nutrient-rich medium, isolated by centrifugation and cultured in water for 10 hours. Culture supernatant was sampled every hour. Optical density (OD₆₀₀) and pH were measured for each sample and molecules in each sample were purified using PGC cartridges and assayed for NOD2 activating activity. This analysis indicated that molecules that activate NOD2 were primarily released after 6 hours in culture (FIG. 7).

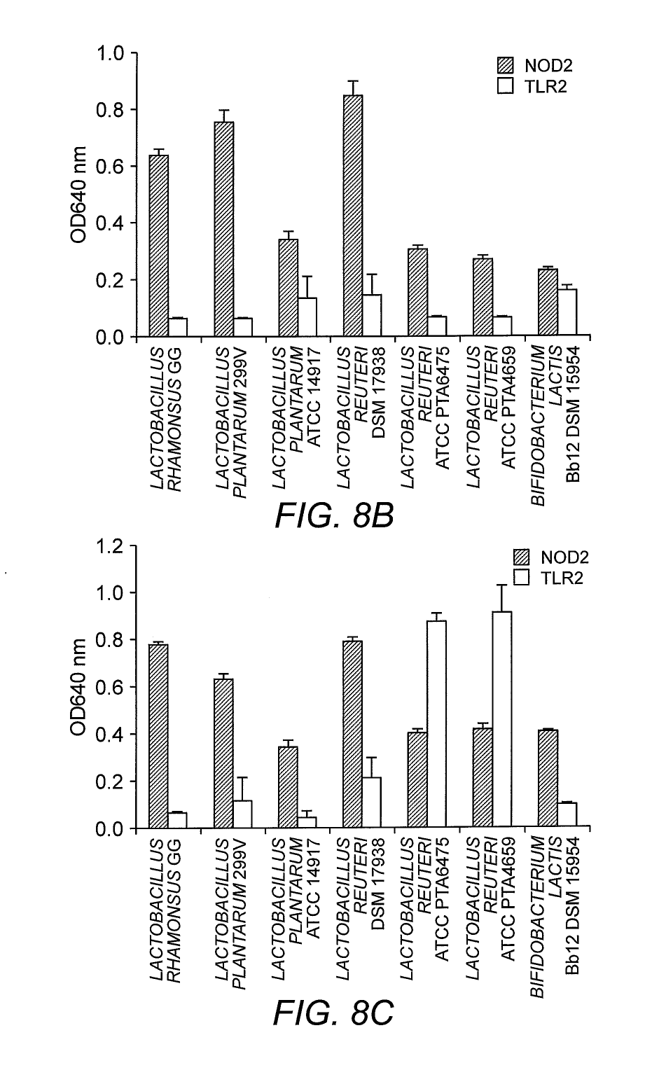

It was further observed that both NOD2 and TLR2 activation could be achieved using crude culture supernatant and that the ability to stimulate NOD2 and TLR2 was dependent on the presence and type of monosaccharide added to the culture medium (FIGS. 8A-8C). In particular, it was observed that small molecules recovered by this method in particular from closely related strains of Lactobacillus had distinct abilities to activate NOD2 and TLR2 in vitro, and this difference was determined by the choice of monosaccharide used in the second cultivation step. For example, different patterns of NOD2 and TLR2 activation by small molecules from closely related strains L. reuteri DSM 17938 and L. reuteri ATCC PTA 6475 were clearly present when the bacteria were cultured in either water (FIG. 8A), water/glucose (FIG. 8B) or water/fructose (FIG. 8C).

By molecular size and composition comparisons it was determined that the small molecules of this invention are distinct from the known NOD2 agonist, muramyl dipeptide (MDP), which is considered the minimal bacterial glycoprotein motif responsible for NOD2 activation. Microbial molecules recovered using the method of the invention are also distinct from known synthetic MDP-derivatives, including the synthetic analogue Mifamurtide, which activates NOD2. In particular, using LC/MS it was found that molecules recovered from strains of L. rhamnosus, L. jensenii, L. crispatus and L. reuteri using the method of this invention have a molecular weight of 348.1 Daltons in the positive ion mode and 346.0 Daltons in the negative ion mode (FIGS. 3A-3D and 4A-4D), which differs from both MDP (molecular weight 492.4 Da) and Milamurtide (molecular weight 1237.5 Da).

It was also shown by glycan analysis that microbial molecules recovered by the instant method from L. rhamnosus GG lack detectable N-acetylglucosamine (GlcNAc) (Table 1), which is a defining molecular component of bacterial peptidoglycan in general, and MDP in particular.

In addition, small molecules recovered via the instant method from L. rhamnosus GG have the ability to modulate MDP activation of NOD2 in vitro further indicating that the small molecules isolated by this method are biologically distinct from MDP.

### Example 5

### Ex Vivo/In Vivo Characterization of Bioactive Small Molecules from Water as Medium

Probiotic bacteria were grown in nutrient rich medium, transferred to water±a monosaccharide and small molecules were isolated from the supernatant as described herein. A range of experiments using ex vivo human tissues and in vivo models were used to demonstrate that the small molecules isolated by the method of this invention have utility in modulating biologic factors associated with immunity, inflammation, infection and cancer, and weight management.

Skin (Ex Vivo).

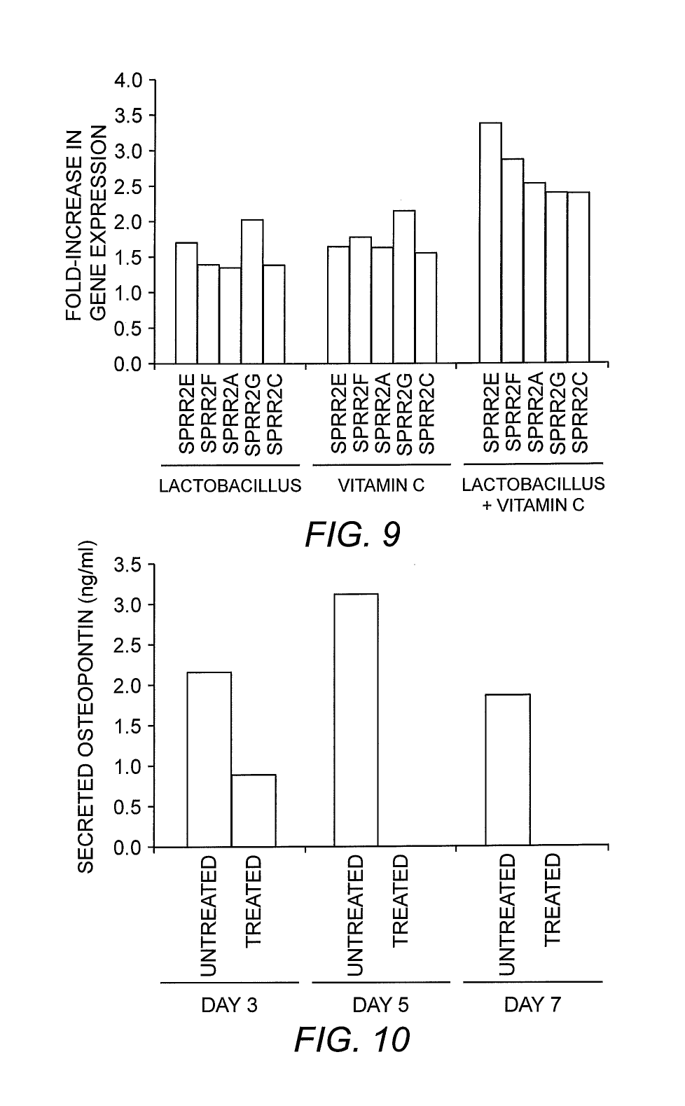

Topical treatment of human full-thickness skin equivalents (MatTek Corp.) with small molecules isolated from L. rhamnosus GG was found to synergize with Vitamin C to boost gene expression of small proline-rich proteins (SPRR) in the skin (FIG. 9). SPRR proteins within the skin function to combat the presence of damaging reactive oxygen species (ROS). Unlike topical application of anti-oxidant agents, SPRR proteins are endogenous within the skin and adapt to the level of ROS, thereby providing a continuous antioxidant barrier. A method for stimulating endogenous SPRR expression in skin using the small molecules of this invention would be considered advantageous for enhancing the protective anti-oxidant barrier of the epidermis.

Female Genital Tract (Ex Vivo).

By RNA microarray analysis, it has been shown that ex vivo treatment of human ectocervical (ECX) tissues with small molecules from multiple different strains of Lactobacillus results in down-regulation of a large gene cluster associated with acute inflammatory responses. More specifically, ex vivo treatment of primary human ECX tissues with small molecules from several different strains of Lactobacillus decreases gene expression of WAP four-disulfide core domain 2 (WFDC2), also called Human epididymis protein 4 (HE4). HE4 is one of only two molecules approved by the FDA as a specific biomarker for epithelial ovarian cancer (EOC). Clinical studies have established that serum HE4 levels are markedly elevated in the majority of patients with EOC, and that elevated serum HE4 correlates with chemoresistance and decreased survival. Currently, no method exists for decreasing HE4 expression in the female reproductive tract. Accordingly, the small molecules of this invention may be of use in decreasing HE4 levels in the treatment of EOC, in particular chemoresistant EOC.

In another example, it was observed that ex vivo treatment of primary human ECX tissues for 1 hour with small molecules isolated from L. rhamnosus GG significantly downregulated expression of genes associated with inflammation. Notably, expression of genes for secretoglobins, matrix metalloproteinase-7 (MMP-7), MMP-9 and secreted phosphoprotein 1 (SPP1, also called Osteopontin) were significantly decreased in tissues from several different donors. Therefore, the small molecules of this invention may be of use in decreasing inflammatory responses in human ectocervical tissues.

Treatment of human ECX tissues with small molecules from L. rhamnosus GG have also been shown to significantly decrease secretion of Osteopontin protein in vitro (FIG. 10). Osteopontin plays a key role in inflammation, cell migration and adhesion, and is overexpressed in a number of human cancers including ovarian cancer. A method for decreasing secretoglobins, MMP7, MMP9 and/or Osteopontin expression in tissues from the female genital tract using small molecules from endogenous strains of Lactobacillus would therefore be considered beneficial in decreasing inflammatory responses and treating ovarian cancer.

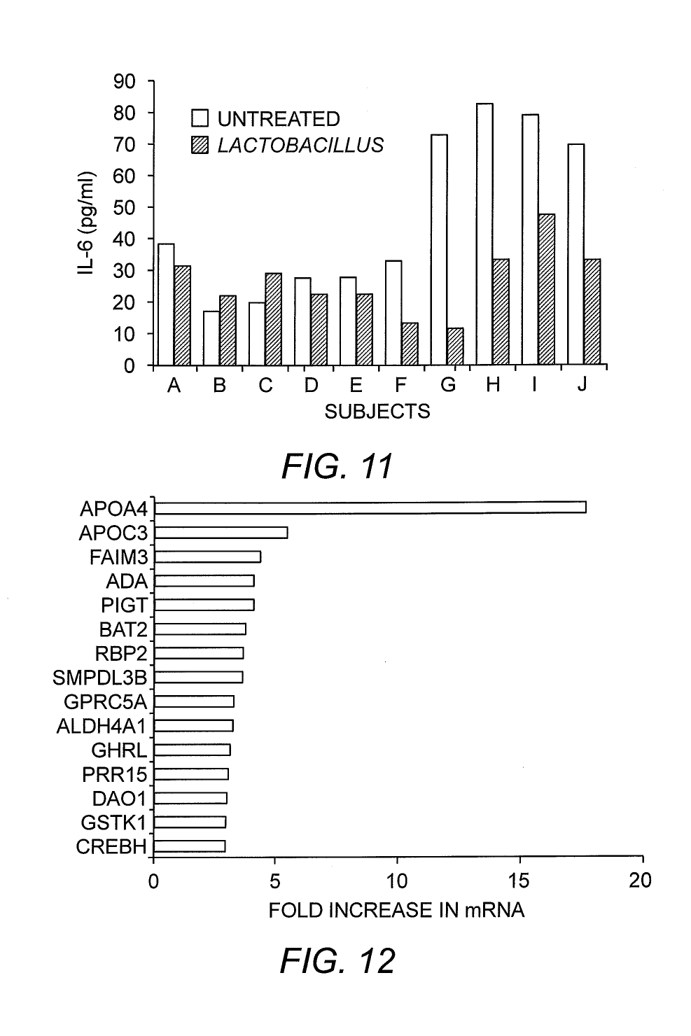

Ex vivo treatment of human ECX tissues with small molecules from L. rhamnosus GG also significantly modulated secretion of IL-6 (FIG. 11). IL-6 is a pleiotropic cytokine that is involved in inflammation. Therapeutic blockade of interleukin-6 using monoclonal antibodies is approved for the treatment of inflammatory diseases. Accordingly, methods to reduce IL-6 secretion in the female reproductive tract using small molecules from endogenous strains of Lactobacillus would likewise be considered beneficial.

In addition, ex vivo treatment of human ECX tissue with small molecules isolated from multiple strains of Lactobacillus decreases replication of HIV-1 in vitro and reduces reverse transcription of viral RNA in HIV-infected tissues by more than 95%. Further, using microarray analysis, it was observed that ex vivo treatment of HIV-infected human ECX tissue with small molecules isolated from two distinct strains of Lactobacillus commonly found in the genital tract of healthy women (L. crispatus and L. jensenii) modulate gene expression associated with cell-cell adhesion, chromatin assembly and cytokine receptor interactions in a strain-specific manner. This result demonstrates that small molecules isolated by this method can be used to distinguish among closely related strains of the same species on the basis of differences in biological activity and that microbial small molecules isolated by the method of the invention have utility in reducing viral infection and replication.

Intestine (Rodent Model).

Small molecules recovered by the method of the invention were found to exhibit biological activity in vivo. Oral administration of small molecules from L. rhamnosus GG to female C57BL/6 mice resulted in significant modulation of gene expression in the small intestine within 30 minutes of intragastric gavage (FIG. 12). Specifically, by RNA microarray analysis, it was shown that exposure to these molecules mediated a 17-fold upregulation in intestinal expression of the gene for Apolipoprotein A-IV (ApoA-IV). ApoA-IV is a pleiotropic molecule that modulates chylomicron formation and lipid efflux from the small intestine, inhibits gastric emptying and serves as a satiety factor. It also possesses anti-inflammatory and antiatherogenic properties. A method for increasing ApoA-IV expression in the small intestine would therefore be considered advantageous for modulating any or all of these known effects.

Continuous oral administration of small molecules from L. rhamnosus GG to female C57BL/6 mice in drinking water for 8 weeks was found to be safe and well tolerated. Oral treatment for 8 weeks yielded significant decreases in the levels of plasma cytokines, specifically CXC19 (MIG), CXCL10 (IP-10) and CCL11 (Table 3). These cytokines serve as immune cell chemoattractants associated with recruitment of cells during infection, allergy and/or inflammation.

TABLE 3

Level (pg/ml)

| | | L. rhamnosus GG |
|---|---|---|
| Cytokine | Water | Small Molecules |
| CXCL9 | ~50 ± 10 | ~40 ± 10 |
| CXCL10 | ~115 ± 15 | ~85 ± 15 |
| CCL11 | ~860 ± 260 | ~600 ± 100 |

Moreover, by RT-PCR analysis it was shown that continuous oral administration of small molecules from L. rhamnosus GG to female C57BL/6 mice in drinking water for 8 weeks leads to a decrease in gene expression of TNFα (1-fold decrease), INFγ (2-fold decrease) and COX2 (2-fold decrease) and an increase iNOS (2-fold increase) and tight junction proteins, Claudin-1 (1-fold increase) and Occludin (10-fold increase), in the small intestine.

Weight Management (Rodent Model).

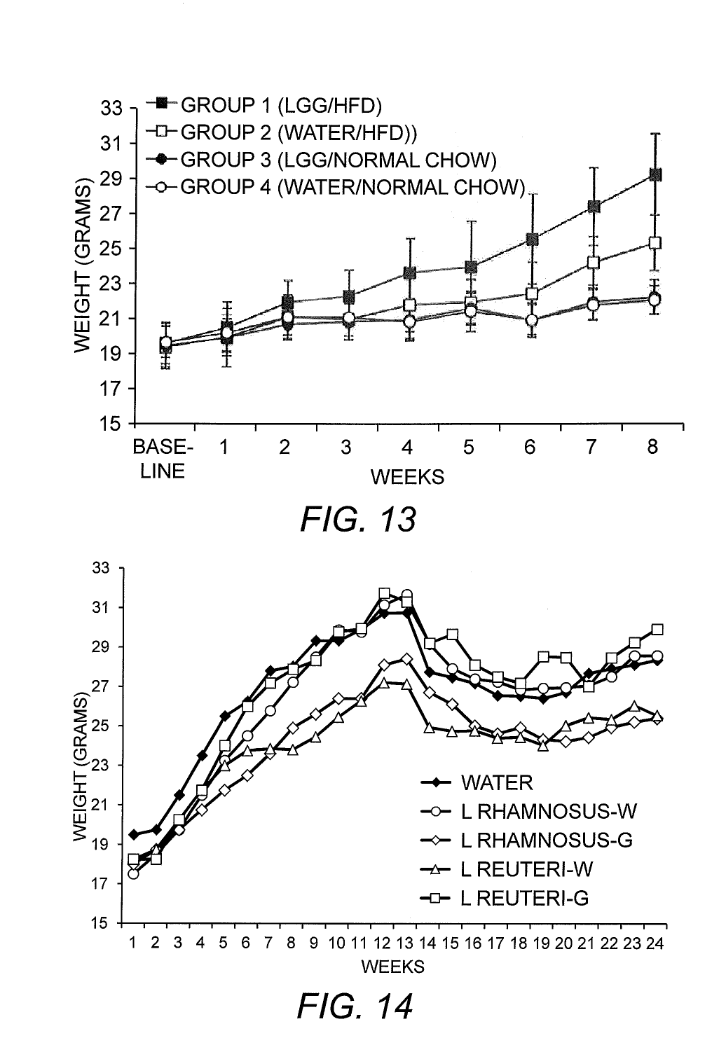

Continuous oral administration of microbial small molecules from L. rhamnosus GG to female C57BL/6 mice in drinking water for 8 weeks, in conjunction with a high fat diet, was found to lead to enhanced weight gain as compared to animals on the same high fat diet given plain drinking water (FIG. 13).

Oral treatment for 8 weeks also yielded significant decreases in the levels of gut hormones in the plasma of mice on a normal diet, specifically ghrelin, gastric inhibitory peptide, insulin and leptin (Table 4). These hormones regulate energy metabolism, appetite and satiety.

TABLE 4

Level (pg/ml)

| | | L. rhamnosus GG |
|---|---|---|
| Hormone | Water | Small Molecules |
| Ghrelin | ~1350 ± 1000 | ~500 ± 100 |
| GIP | ~200 ± 180 | ~60 ± 30 |
| Insulin | ~1200 ± 1000 | ~150 ± 60 |
| Leptin | ~4300 ± 4000 | ~1200 ± 450 |

FIG. 14 shows that oral administration to C57BL/6 female mice of crude filtered supernatants of L. rhamnosus or L. reuteri cultured in either water (W) or glucose (G) according to the method of the invention for 24 weeks resulted in reduced weight gain in the first 12 weeks on a Western Diet (high in fat and sugar) and enhanced weight loss in the second 12 weeks on a normal diet.

This finding demonstrates that microbial small molecules isolated by this method have effects on energy uptake and homeostasis, and are useful in the treatment of conditions associated with weight management.

What is claimed is:

1. A method for isolating bioactive small molecules from probiotic bacteria comprising
(a) culturing pure probiotic bacteria in nutrient-rich medium for approximately 18 to 24 hours;
(b) separating the probiotic bacteria from the nutrient-rich medium;
(c) washing the probiotic bacteria with water to remove residual nutrient-rich medium;
(d) culturing the probiotic bacteria in a second medium consisting of water or water and a monosaccharide for approximately seven to 24 hours;
(e) separating the probiotic bacteria from the second medium to yield a bacteria-free supernatant containing bioactive small molecules;
(f) applying the bacteria-free supernatant to an activated porous graphitized carbon (PGC) matrix;
(g) washing the PGC matrix to remove unbound material; and
(h) eluting bound bioactive small molecules by sequential application of
(i) a polar solvent and
(ii) a polar solvent with an acidic modifier thereby isolating bioactive small molecules secreted into a bacterial culture medium.

2. The method of claim 1, wherein the probiotic bacterium is selected from the genera of Lactobacillus, Streptococcus, Enterococcus and Bifidobacterium.

3. The method of claim 1, wherein the polar solvent comprises acetonitrile.

4. The method of claim 1, wherein the polar solvent of (i) comprises application of 10% acetonitrile followed by application of 25% acetonitrile.

5. The method of claim 1, wherein the acidic modifier comprises, acetic acid, formic acid or trifluoroacetic acid.

6. The method of claim 1, wherein the polar solvent with an acidic modifier of (ii) comprises sequential application of 25% acetonitrile and 0.1% acetic acid; 25% acetonitrile and 0.1% formic acid; and 25% acetonitrile and 0.05% trifluoroacetic acid.

7. A bioactive small molecule isolated by the method of claim 1.

8. A method for activating NOD2 comprising contacting a cell with the isolated bioactive molecule of claim 7 thereby activating NOD2.

9. A method for decreasing expression of inflammatory molecules comprising contacting a cell with the isolated bioactive molecule of claim 7 thereby decreasing expression of inflammatory molecules by the cell.

10. The method of claim 9, wherein the inflammatory molecules comprise secretoglobins, matrix metalloproteinase-7 (MMP-7), MMP-9, osteopontin, IL-6, CXCL9, CXCL10 and CCL11.

11. A method for inhibiting replication of human immunodeficiency virus (HIV) comprising contacting HIV-uninfected or HIV-infected cells with the isolated bioactive molecule of claim 7 thereby inhibiting the infection or replication of HIV.

12. A pharmaceutical composition comprising the isolated bioactive molecule of claim 7 in admixture with a pharmaceutically acceptable carrier.

13. The pharmaceutical composition of claim 12, further comprising vitamin C.

14. A method for stimulating expression of Apolipoprotein A-IV administering to a subject in need thereof an effective amount of the pharmaceutical composition of claim 12 thereby stimulating expression of Apolipoprotein A-IV.

15. A method for modulating diet-associated weight gain comprising administering to a subject in need thereof an effective amount of the pharmaceutical composition of claim 12 thereby modulating diet-associated weight gain of the subject.

16. A method for preventing mucosal transmission of human immunodeficiency virus (HIV) comprising administering to the mucosa of a subject an effective amount of the pharmaceutical composition of claim 12, thereby preventing mucosal transmission of HIV to the subject.

17. The method of claim 16, wherein the pharmaceutical composition is formulated in an acid-buffering gel or cream for topical administration to the skin, vaginal surface or gastrointestinal surface.

18. The method of claim 16, wherein the pharmaceutical composition is formulated for oral administration to an infant exposed to HIV-1 through breastfeeding.
